SEQUENCE LISTING (5 sequences)

SEQ ID NO: 1 - protein, 37 aa, Artificial
  synthetic construct
KCNTATCATQRLANFLVHSSNNFGPILPPTNVGSNTY

SEQ ID NO: 2 - protein, 12 aa, Artificial
  synthetic construct
ANFLVHSSNNFG

SEQ ID NO: 3 - protein, 13 aa, Artificial
  synthetic construct
PILPPTNVGSNTY

SEQ ID NO: 4 - protein, 25 aa, Artificial
  synthetic construct
ANFLVHSSNNFGPILPPTNVGSNTY

SEQ ID NO: 5 - protein, 12 aa, Artificial
  synthetic construct
KCNTATCATQRL

(19) United States
(12) Patent Application Publication
(10) Pub. No.: US 2010/0249370 A1
(43) Pub. Date: Sep. 30, 2010

(54) PROCESS FOR THE PRODUCTION OF PRAMLINTIDE

(75) Inventors: Andreas Brunner, Brig (CH); Oleg Werbitzky, Veyras (CH); Stephane Varray, Sierre (CH); Francesca Quattrini, Zurich (CH); Holger Hermann, Visp (CH); Andrew Strong, Vevey (CH); Fernando Albericio, Barcelona (ES); Judit Tulla-Puche, Girona (ES); Yesica Garcia Ramos, Barcelona (ES)

Correspondence Address: HOFFMANN & BARON, LLP, 6900 JERICHO TURNPIKE, SYOSSET, NY, 11791, US

(73) Assignee: LONZA AG, Basel (CH)

(21) Appl. No.: 12/665,844

(22) PCT Filed: Jun. 30, 2008
(86) PCT No.: PCT/EP2008/005325

§ 371 Date: Dec. 21, 2009

(30) Foreign Application Priority Data
Jun. 29, 2007 (EP) EP07012806
Nov. 19, 2007 (EP) EP07022380

(51) Int. Cl. C07K 7/50; C07K 7/08; C07K 14/00

(52) U.S. Cl. 530/317; 530/327; 530/324

(57) ABSTRACT

Pramlintide, a peptide having the 37 amino acid sequence KCNTATCATQRLANFLVHSSNNFGPILPPT-NVGSNTY-NH2 is prepared via a convergent three-fragment synthesis strategy from the fragments comprising the amino acid residues 1-12, 13-24 and 25-37, respectively.

The invention relates to a novel convergent synthesis of pramlintide which is a 37-mer peptide of formula

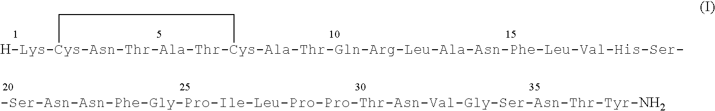

The invention further relates to several side chain-protected peptides as intermediates in the synthesis of pramlintide.

Pramlintide (25,28,29-pro-h-amylin; Chem. Abstr. Reg. No. 151126-32-8) is an antidiabetic analogue of human amylin that is marketed by Amylin Pharmaceuticals, Inc., under the brand name Symlin® (WO-A-93/10146).

Known syntheses of amylin and amylin analogues (WO-A-93/10146, U.S. Pat. No. 5,424,394) are using a classical stepwise approach. Single amino acid residues are covalently coupled to a growing peptide chain which is covalently linked to a solid resin support (SPPS). The intramolecular disulfide bond between the cysteine residues at positions 2 and 7 of the peptide chain is formed after completion of the peptide chain, but before the peptide is cleaved off the resin. That synthetic route is very lengthy and somewhat inefficient since several coupling steps have to be repeated in order to achieve satisfactory coupling yields (U.S. Pat. No. 5,424,394).

Generally, convergent synthesis is an alternative approach for assembling peptides, which applies to pramlintide of course as well (WO-A-2006/045603). The challenge of convergent synthesis is to find suitable fragments and their coupling order for overcoming the known drawbacks of convergent synthesis. These drawbacks are solubility problems during coupling and purification, lower reaction rates compared to SPPS and a much higher racemization risk of the C terminal fragment during coupling. Pramlintide consists of thirty-seven amino acid residues so that a huge number of possible fragments and coupling orders exists. However, prior art, and specifically WO-A-2006/045603, is silent concerning a concrete selection of suitable fragments and coupling orders.

It is an object of the present invention to provide a more efficient synthesis of pramlintide that overcomes the known drawbacks of convergent synthesis and is suitable for the production on an industrial scale. This object has been achieved by the synthesis according to claim 1 and the peptide fragments of claims 10 to 21.

After several, unsuccessful experiments of different fragment coupling strategies (see comparison examples), applicants have surprisingly found a suitable strategy which comprises the specific coupling of three peptide fragments, one of them containing the two cysteine residues with pre-formed disulfide bond. In particular, the process of the invention comprises the steps of

(a) reacting a side chain-protected peptide of formula

P-Ala-Asn-¹⁵Phe-Leu-Val-His-Ser-²⁰Ser-Asn-Asn-Phe-Gly-OH (II)
wherein P is a protecting group being orthogonal to the side chain protecting groups, with a side chain-protected peptide of formula

H-²⁵Pro-Ile-Leu-Pro-Pro-³⁰Thr-Asn-Val-Gly-Ser-²⁵Asn-Thr-Tyr-NH₂ (III)
to yield a side-chain protected peptide of formula

P-Ala-Asn-¹⁵Phe-Leu-Val-His-Ser-²⁰Ser-Asn-Asn-Phe-Gly-²⁵Pro-Ile-Leu-Pro-Pro-³⁰Thr-Asn-Val-Gly-Ser-³⁵Asn-Thr-Tyr-NH₂ (IV)
wherein P is as defined above,
   (b) removing the terminal P protecting group of the peptide produced in (a)
   (c) reacting the peptide produced in (b) with a side chain-protected peptide of formula

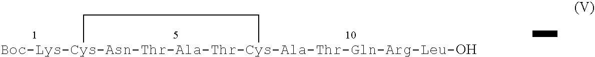

to yield side-chain protected pramlintide with Boc-protected amino terminus, and deprotecting the side chains and the amino terminus of the product obtained in (c) to yield pramlintide (I).

Here and in the following, the term “a protecting group being orthogonal to the side chain protecting groups” is to be understood to mean a protecting group which may be cleaved by a method that does not affect the side chain protecting groups.

Preferably, the protecting group P is fluoren-9-ylmethoxycarbonyl (Fmoc) or 2-(4-nitrophenyl-sulfonyl)ethoxycarbonyl (NSC).

Most preferably, the process of the invention comprises the steps of

(a) reacting a side chain-protected peptide of formula

Fmoc-Ala-Asn-¹⁵Phe-Leu-Val-His-Ser-²⁰Ser-Asn-Asn-Phe-Gly-OH
with a side chain-protected peptide of formula

H-²⁵Pro-Ile-Leu-Pro-Pro-³⁰Thr-Asn-Val-Gly-Ser-³⁵Asn-Thr-Tyr-NH₂ (III)
to yield a side chain-protected peptide of formula

Fmoc-Ala-Asn-¹⁵Phe-Leu-Val-His-Ser-²⁰Ser-Asn-Asn-Phe-Gly-²⁵Pro-Ile-Leu-Pro-Pro-³⁰Thr-Asn-Val-Gly-Ser-³⁵Asn-Thr-Tyr-NH₂

(b) removing the terminal Fmoc protecting group of the peptide produced in (a)
(c) reacting the peptide produced in (b) with a side chain-protected peptide of formula

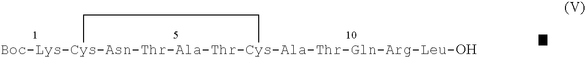

to yield side chain-protected pramlintide with Boc-protected N-terminus, and
   (d) deprotecting the side chains and the N-terminus of the product obtained in (c) to yield pramlintide (I).

Steps (a) to (d) can be carried out using reaction conditions known in the art of peptide synthesis.

The coupling and deprotection steps (a), (b) and (c) are suitably performed in solution, preferably in N,N-dimethylformamide (DMF).

The combination of 6-chloro-1-hydroxybenzotriazole (6-Cl—HOBt), 5-chloro-1-[bis(dimethyl-amino)methylene]-1H-benzotriazolium 3-oxide tetrafluorophosphate (TCTU) and diisopropyl-ethylamine (DIEA) is preferably used as coupling agent in steps (a) and (c).

The removal of the Fmoc protecting group of the intermediate coupling product IV in step (b) is preferably accomplished with piperidine in DMF.

The final deprotection step (d) is preferably carried out with trifluoroacetic acid, triisopropyl-silane and phenol.

In a preferred embodiment, one or more of the Ser and Thr residues in the peptide fragments (II), (III) and (V) are present as pseudoproline derivatives. These pseudoproline moieties improve the solubility of the peptides and prevent or decrease aggregation.

The crude product obtained after step (d) can be purified by conventional methods, e.g. with preparative HPLC or countercurrent distribution. The same applies to the intermediates obtained after steps (a), (b) and (c), if purification is required.

The side chain-protected peptide fragments (II), (III) and (V) can be prepared using conventional peptide synthesis methods, e.g. solution-phase synthesis (SPS) or solid-phase synthesis (SPPS). In case of SPPS, all resins being known to the person skilled in the art and allowing the preparation of protected peptides can be applied. Here, resins are to be interpreted in a wide manner. Therefore, the term “resin” is to be understood to mean e.g. a solid support alone or a solid support directly linked to a linker, optionally with a handle in between. Preferred resins are polystyrene-based resins with trityl, bromobenzhydryl, Sieber amide or xanthenyl amide (XAL) linkers. Examples for trityl resins are 2-chlorotrityl chloride resin (CTC resin), trityl chloride resin, 4-methyltrityl chloride resin and 4-methoxytrityl chloride resin. Examples for Sieber amide resins are 9-Fmoc-aminoxanthen-3-yloxy-Merrifield resin (Sieber resin) and 9-Fmoc-aminoxanthen-3-yloxy TG resin (NovaSyn® TG Sieber resin). Preferably, the CTC resin and the Sieber resin are applied, and most preferably the CTC resin is applied for the synthesis of fragments containing the free carboxylic function and the Sieber resin for the preparation of the C-terminal fragment ending by the tyrosine amide.

The disulfide bridge in peptide fragment (V) is suitably formed while the fragment is still attached to the resin. Pseudoproline units can be introduced by using the commercially available pseudoproline dipeptides instead of single Ser or Thr units with conventional side chain-protecting groups.

Another object of the invention is to provide side chain-protected peptides which are useful as intermediates in the process of the invention. In particular, one of these peptides is a side chain-protected peptide of formula

P-Ala-Asn-Phe-Leu-Val-His-Ser-Ser-Asn-Asn-Phe-Gly-OH (II)

wherein P is a protecting group being orthogonal to the side chain protecting groups.

Preferably, the peptide of formula (II) has a side chain-protection scheme of

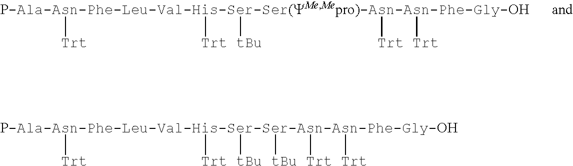

wherein P is as defined above.

Preferably, the protecting group P is Fmoc or NSC.

Even more preferably, P is Fmoc, thus affording the following side chain-protected peptide

Fmoc-Ala-Asn-Phe-Leu-Val-His-Ser-Ser-Asn-Asn-Phe-Gly-OH

most preferably having a side chain-protection scheme of

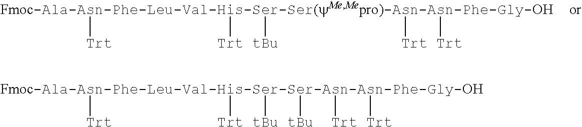

and comprising the amino acids Nos. 13-24 of pramlintide.

In particular, the others of said side chain-protected peptides being useful as intermediates in the process of the invention is a side chain-protected peptide of formula

H-Pro-Ile-Leu-Pro-Pro-Thr-Asn-Val-Gly-Ser-Asn-Thr-Tyr-NH₂ (III)

preferably having a side chain-protection scheme of

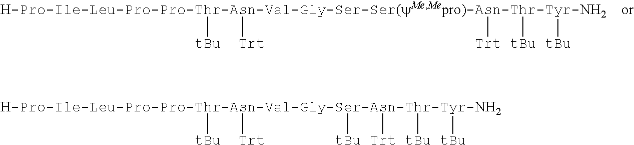

and comprising the amino acids Nos. 25-37 of pramlintide;
and a side chain-protected peptide of formula

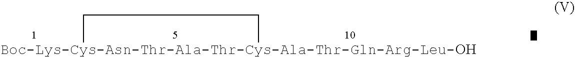

preferably having a side chain-protection scheme of

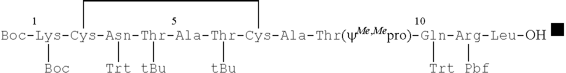

and comprising the amino acids Nos. 1-12 of pramlintide;
and a side chain-protected peptide of formula

R-Ala-Asn-Phe-Leu-Val-His-Ser-Ser-Asn-Asn-Phe-Gly-Pro-Ile-Leu-Pro-Pro-Thr-Asn-Val-Gly-Ser-Asn-Thr-Tyr-NH₂

wherein R is hydrogen or a protecting group P that is orthogonal to the side chain protecting groups,
preferably having one of the side chain-protection schemes of

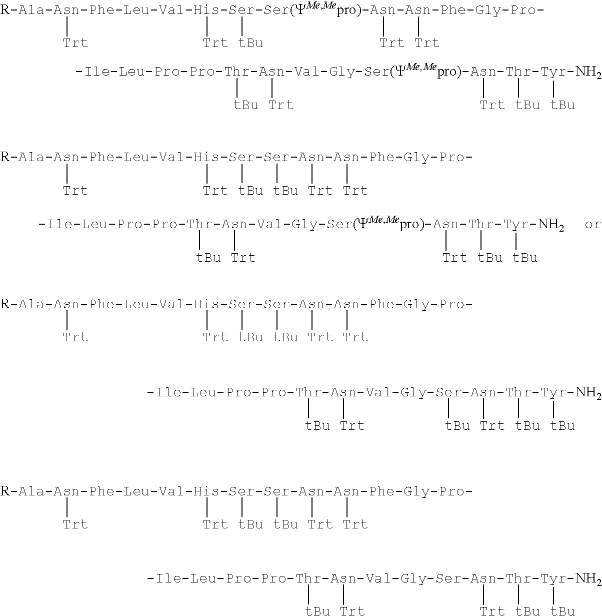

wherein R is as defined above and comprising the amino acids Nos. 13-37 of pramlintide. Preferably, R is the protecting group P. More preferably, P is selected from the group consisting of Fmoc and NSC; and most preferably, P is Fmoc.

### EXAMPLES

The following non-limiting examples will illustrate representative embodiments of the invention in detail.

### Abbreviations:

CTC resin=2-chlorotrityl chloride on polymeric support
DCM=dichloromethane
DIC=diisopropylcarbodiimide
DIEA=diisopropylethylamine
HCTU=2-(6-chloro-1H-benzotriazol-1-yl)-1,1,3,3-tetramethyluronium hexafluorophosphate
TCTU=5-chloro-1-[bis(dimethylamino)methylene]-1H-benzotriazolium 3-oxide tetrafluoro-phosphate
HOBt=1-hydroxybenzotriazole
6-Cl—HOBt=6-chloro-1-hydroxybenzotriazole
Pbf=2,2,4,6,7-pentamethyldihydrobenzofuran-5-sulfonyl
TFA=trifluoroacetic acid

### Example 1

### Synthesis of

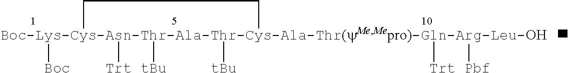

The peptide was synthesized on CTC resin using Fmoc-protected amino acids with the respective side chain-protecting groups, if applicable. For the last coupling step, Boc-Lys(Boc)-OH was used. Amino acid Nos. 8 and 9 were employed as pseudoproline dipeptide Fmoc-Ala-Thr(Ψ^{Me,Me}pro)-OH which is commercially available from Merck Biosciences under the Novabiochem brand or from Genzyme Pharmaceuticals. Cysteine was used as the Fmoc-Cys(Acm)-OH (Acm=acetamidomethyl) derivative. The synthesis was carried out on 150 g of CTC resin with a loading of 0.64 mmol/g and 2.5 equivalents of each amino acid. The amino acids were coupled using a TCTU/6-Cl—HOBt/DIEA reagent mixture. Each amino acid was pre-activated in a separate vessel at 0-5° C. and transferred into the peptide synthesizer where the coupling step was performed at 20° C. The completeness of each coupling step was monitored by the Kaiser test and by HPLC. It appeared that no coupling step had to be repeated. The cleavage of the Fmoc protecting group was accomplished by treating the elongated peptide three times with piperidine (20 wt. %) in N-methylpyrrolidinone (PIP/NMP) at 30° C. After the last elongation cycle the peptide-loaded resin was washed three times with NMP and flushed with nitrogen. The disulfide bond was formed within 15 min at 0° C. using 3 equivalents of iodine in DMF. After washing several times with pure DMF the resin was treated three times with 1% trifluoroacetic acid in dichloromethane to cleave off the peptide. A yellow oil was obtained after evaporation of the dichloromethane. The peptide was precipitated in water, filtered and dried to yield 222.9 g of a white powder.

### Example 2

### Synthesis of

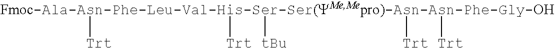

The synthesis was performed in a similar way as described in Example 1, using 80 g of CTC resin to which the C-terminal amino acid (Fmoc-Gly-OH) of the fragment was attached to give 0.66 mmol/g loading. The chain elongation was performed with 2.2 to 2.5 equivalents of Fmoc-protected amino acids. The coupling step of the Phe residue next to the C-terminus was repeated once, using DIC/6-Cl—HOBt activation (2.5 equivalents) while a single coupling step was sufficient for each remaining amino acid. The pseudoproline unit was introduced using the Fmoc-Ser(tBu)-Ser(Ψ^{Me,Me}pro)-OH dipeptide building block. After completion of the elongation steps the protected peptide was cleaved from the resin in two batches using 2% trifluoro-acetic acid in dichloromethane. The combined cleavage solutions were concentrated in vacuo and the peptide was precipitate with water, filtered and dried to yield 143 g of crude peptide. The corresponding peptide containing a Ser(tBu) unit instead of the Ser(Ψ^{Me,Me}pro) pseudoproline was prepared analogously, using two Fmoc-Ser(tBu)-OH building blocks instead of the Fmoc-Ser(tBu)-Ser(Ψ^{Me,Me}pro)-OH dipeptide (see Example 10).

### Example 3

### Synthesis of

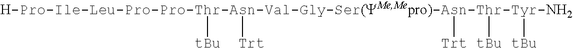

The peptide was synthesized on Sieber resin (150 g, 0.55 mmol/g loading) using standard Fmoc chemistry. The pseudoproline unit was introduced using the Fmoc-Gly-Ser(Ψ^{Me,Me}pro)-OH dipeptide as building block. The coupling and Fmoc deprotection steps were carried out as described in Example 1. The peptide cleavage from the resin was performed using 3% trifluoroacetic acid in dichloromethane. The peptide-loaded resin was treated with the TFA/DCM solution five times to give a yellow oil after evaporation of the solvent. The peptide was precipitated in water, filtered and dried to yield 145.75 g of a white powder.

The corresponding peptide containing a Ser(tBu) unit instead of the Ser(Ψ^{Me,Me}pro) pseudoproline was prepared analogously, using Fmoc-Ser(tBu)-OH and Fmoc-Gly-OH instead of the Fmoc-Gly-Ser(Ψ^{Me,Me}pro)-OH dipeptide (see Example 9).

Cleavage with 5% trifluoroacetic acid in dichloromethane resulted in the formation of the corresponding peptide with unprotected Ser side chain.

### Example 4

### Synthesis of

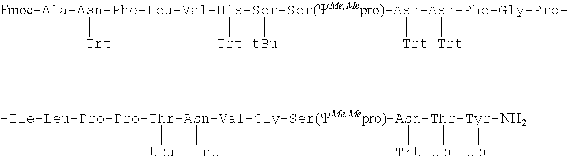

The Fmoc-protected peptide obtained in Example 2 (4.57 g) was pre-activated with 6-Cl—HOBt (0.31 g), TCTU (0.63 g) and DIEA (0.60 g) in DMF (52 g) for 10 min, then the peptide obtained in Example 3 (3.80 g) and further DIEA (0.78 g) were added to effect the fragment coupling reaction. After 0.5 h, extra 6-Cl—HOBt (0.03 g) and TCTU (0.06 g) were added and the reaction mixture was allowed to warm up to 20° C. The reaction mixture was worked up by cooling to 0-5° C., precipitation of the peptide in aqueous solution, filtration and washing. Yield: 7.63 g.

The corresponding peptides wherein one or both pseudoproline units are replaced with Ser(tBu) or wherein the pseudoproline unit near the N-terminus is replaced with Ser(tBu) and the pseudoproline unit near the C-terminus is replaced with unprotected Ser were prepared in an analogous way.

### Example 5

### Synthesis of

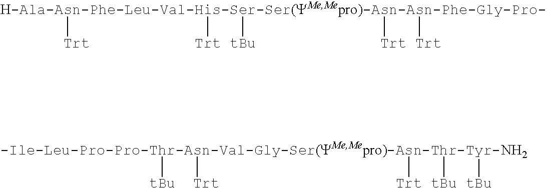

The Fmoc-protected peptide obtained in Example 4 (7.54 g) was dissolved in DMF (25.1 mL) and warmed to 40° C. Piperidine (0.44 g) was added to cleave off the Fmoc protecting group. After 2 h the solution was cooled to 15° C. and water (50 mL) was added to precipitate the product which was isolated by filtration. The wet product was washed three times with DMF/EtOH/water (25.1 mL/12.5 mL/62.6 mL), twice with water (50 mL) and twice with water/EtOH (1:1 v:v, 50 mL).

Yield: 7.03 g.

The Fmoc protecting group of the corresponding peptides wherein one or both pseudoproline units are replaced with Ser(tBu) or wherein the pseudoproline unit near the N-terminus is replaced with Ser(tBu) and the pseudoproline unit near the C-terminus is replaced with unprotected Ser side chain was cleaved in an analogous way.

### Example 6

### Synthesis of

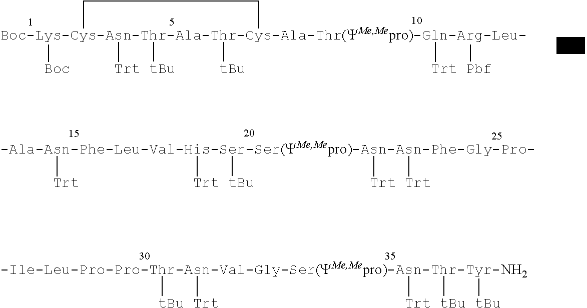

The side chain-protected peptides obtained in Examples 1 (3.73 g) and 5 (6.87 g) were dissolved in DMF (80 mL) and 6-Cl—HOBt (0.26 g) was added. The mixture was cooled to 0° C. and TCTU (0.55 g) and DIEA (0.96 mL) were added. After 1 h the reaction mixture was allowed to assume room temperature and stirring continued for another 3 h. The reaction mixture was cooled to 0° C. and the product was precipitated by addition of water (150 mL). The precipitated peptide was separated by filtration and washed with water (2×75 mL).

Yield: 10.87 g.

The corresponding peptides with only one or two pseudoproline units were prepared in an analogous way from the respective fragments mentioned above.

### Example 7

### Synthesis (Deprotection) of Pramlintide

The protected pramlintide obtained in Example 6 (10.65 g) was dissolved in a mixture of trifluoroacetic acid (101.2 mL), triisopropylsilane (2.66 mL) and phenol (2.66 g) and stirred at 20° C. for 4 h. The deprotected peptide was precipitated by addition of diisopropyl ether (530 mL), stirring was continued for 40 min and the product was separated by filtration on a G3 frit.

Yield: 7.5 g of crude pramlintide.

The crude product was purified by preparative HPLC on Kromasil® 100-10-C18 (20×2.5 cm column, flow rate 30 mL/min). In a first step the crude peptide was purified by gradient elution with acetonitrile/0.2 M triethylammonium phosphate (pH 2.2) at a column loading of 20 mg crude peptide/mL. The product-containing fractions were diluted with an equal volume of water and further purified by gradient elution from the same column (acetonitrile/1% acetic acid, column loading 8 mg peptide/mL). The eluate fractions containing pure product were concentrated in vacuo, filtered and lyophilized to obtain pramlintide with 97.5% purity.

### Example 8

### Synthesis of

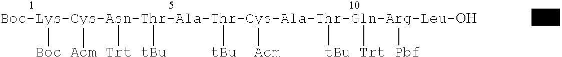

The target compound of Example 8 is the intermediate of Example 1 before cyclization with the exception that no pseudoproline dipeptide was employed. The synthesis was performed on small scale analogous to Example 1 with the exception of Fmoc-⁹Thr(tBu)-OH, Fmoc-⁸Ala-OH and HCTU/DIEA as coupling mixture, yielding the target compound with 57% purity.

### Example 9

### Synthesis of

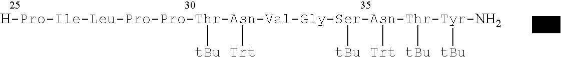

The synthesis was performed on small scale analogous to Example 3 with the exception of Fmoc-³³Gly-OH and Fmoc-³⁴Ser(tBu)-OH instead of the corresponding pseudoproline dipeptide and with the exception of HCTU/DIEA instead of TCTU/6-Cl—HOBt/DIEA as coupling mixture, yielding the target compound with 60% purity.

### Example 10

### Synthesis of

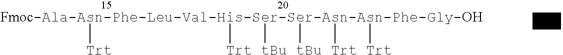

The synthesis was performed analogous to Example 2 with the exception of Fmoc-Ser(tBu)-OH for positions 19 and 20 instead of the corresponding pseudoproline dipeptide, yielding the target compound with 93% purity.

### Comparison Examples C1-C3

### Coupling Strategies of Different Fragments

| | | Reaction conditions and | |
|---|---|---|---|
| No | Fragments | observations | Result |
| C1 | Boc-[1-24]-OH (A) and | For reaction conditions, see⁽¹⁾. | Strategy failed, as no |
| | H-[25-37]-NH₂ (B) | Coupling was | formed (A) could be |
| | | difficult for positions 22, | detected (by HPLC-MS). |
| | | 21 and 9 (by ninhydrin | |
| | | test). | |
| C2 | Boc-[1-20]-OH (A) and | For reaction conditions, | Strategy failed, as no |
| | H-[21-37]-NH₂ (B) | see⁽¹⁾. Coupling was | formed (A) could be |
| | | difficult for positions 5 | detected (by HPLC-MS). |
| | | and 4 (by ninhydrin test). | |
| C3 | Boc-[1-12]-OH (A), | For reaction conditions, | Strategy failed, as the |
| | Fmoc-[13-20]-NH₂ (B) and | see⁽²⁾. Coupling was | precursor Fmoc-(C) was |
| | H-[21-37]-NH2 (C) | difficult for positions 28, | formed with only 11% |
| | | 27 and 22 (by ninhydrin | purity (by HPLC). |
| | | test). | |

⁽¹⁾Analogous to Example 1; single protected amino acids, except for Fmoc-¹⁹Ser--²⁰Ser(ψ^{Me,Me}pro)-OH in Comparison Example C2; HCTU/DIEA as coupling mixture; cyclization after fragment coupling.
⁽²⁾Coupling order: (A) + [(B) + (C)]. Coupling conditions: Analogous to Example 3; single protected amino acids except for Fmoc-¹⁹Ser-²⁰Ser(ψ^{Me,Me}pro)-OH, HCTU/DIEA as coupling method; cyclization on resin.

1. A process for the production of pramlintide of formula 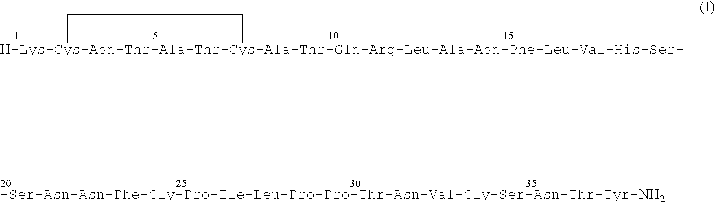
comprising:
(d) reacting a side chain-protected peptide of formula
P-Ala-Asn-¹⁵Phe-Leu-Val-His-Ser-²⁰Ser-Asn-Asn-Phe-Gly-OH (II)
wherein P is a protecting group being orthogonal to the side chain protecting groups, with a side chain-protected peptide of formula
H-²⁵Pro-Ile-Leu-Pro-Pro-³⁰Thr-Asn-Val-Gly-Ser-³⁵Asn-Thr-Tyr-NH₂ (III)
to yield a side-chain protected peptide of formula
P-Ala-Asn-¹⁵Phe-Leu-Val-His-Ser-²⁰Ser-Asn-Asn-Phe-Gly-²⁵Pro-Ile-Leu-Pro-Pro-³⁰Thr-Asn-Val-Gly-Ser-³⁵Asn-Thr-Tyr-NH₂ (IV)
wherein P is as defined above,
(e) removing the terminal P protecting group of the peptide produced in (a)
(f) reacting the peptide produced in (b) with a side chain-protected peptide of formula 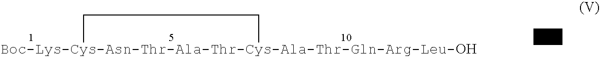
to yield side-chain protected pramlintide with Boc-protected amino terminus, and
(g) deprotecting the side chains and the amino terminus of the product obtained in (c) to yield pramlintide (I).

2. The process of claim 1, wherein P is Fmoc.

3. The process of claim 1, wherein P is NSC.

4. The process of claim 1, wherein steps (a) to (c) are performed in solution.

5. The process of claim 4, wherein N,N-dimethylformamide is used as solvent.

6. The process of claim 1, wherein the coupling steps (a) and (c) are accomplished with a combination of 6-chloro-1-hydroxybenzotriazole, 5-chloro-1-[bis(di-methylamino)]methylene]-1H-benzotriazolium 3-oxide tetrafluorophosphate and diiso-propylethylamine.

7. The process of claim 1, wherein the deprotecting step (b) is carried out with piperidine in DMF.

8. The process of claim 1, wherein the final deprotecting step (d) is carried out with a mixture of trifluoroacetic acid, triisopropylsilane and phenol.

9. The process of claim 1, wherein at least one of peptide fragments (II), (III) and (V) comprises a pseudoproline moiety at one of its Ser or Thr residues.

10. A side chain-protected peptide of formula
P-Ala-Asn-Phe-Leu-Val-His-Ser-Ser-Asn-Asn-Phe-Gly-OH (II)
wherein P is a protecting group being orthogonal to the side chain protecting groups.

11. The peptide of claim 10, which is selected from the group consisting of 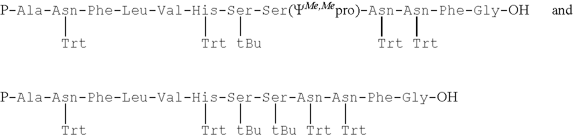
wherein P is as defined in claim 10.

12. The peptide of claim 10, wherein P is Fmoc. 30

13. The peptide of claim 10, wherein P is NSC.

14. A side chain-protected peptide of formula
H-Pro-Ile-Leu-Pro-Pro-Thr-Asn-Val-Gly-Ser-Asn-Thr-Tyr-NH₂ (III)

15. The peptide of claim 14, which is selected from the group consisting of

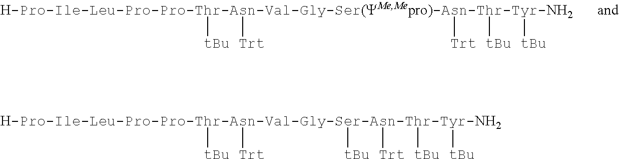

16. A side-chain protected peptide of formula

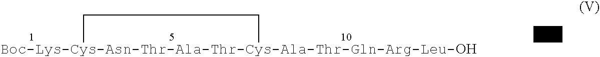

17. The peptide of claim 16, which is

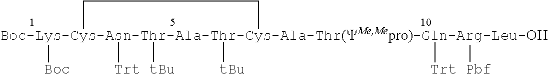

18. A side-chain protected peptide of formula
R-Ala-Asn-Phe-Leu-Val-His-Ser-Ser-Asn-Asn-Phe-Gly-Pro-Ile-Leu-Pro-Pro-Thr-Asn-Val-Gly-Ser-Asn-Thr-Tyr-NH₂
wherein R is hydrogen or a protecting group P that is orthogonal to the side chain protecting groups.

19. The peptide of claim 18, which is selected from the group consisting of 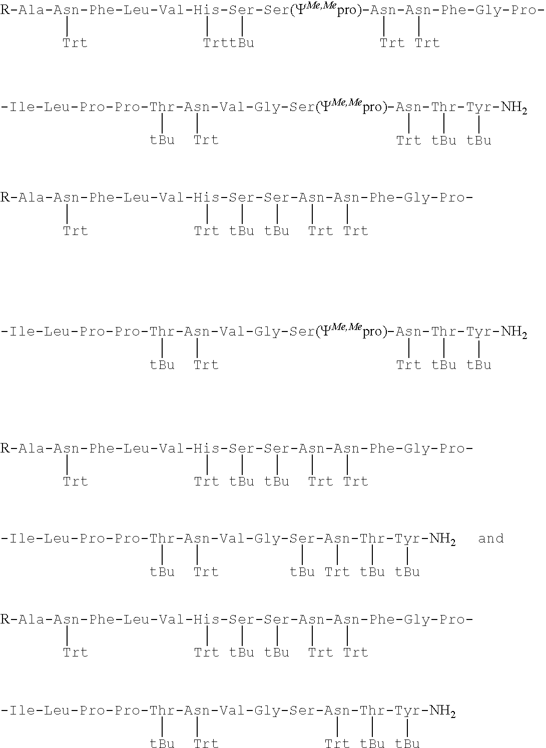
wherein R is as defined in claim 18.

20. The peptide of claim 18, wherein R is the protecting group P.

21. The peptide of claim 20, wherein P is selected from the group consisting of Fmoc and NSC.

22. Use of any one of the peptides of claim 9 as intermediates in a synthesis of pramlintide.
